(19) United States
(12) Patent Application Publication
(10) Pub. No.: US 2009/0011045 A1
(43) Pub. Date: Jan. 8, 2009

(54) Pharmaceutical for Hygienic Administration in the Ear

(75) Inventors: Dirk Mertin, Langenfeld (DE); Gert Daube, Engelskirchen (DE); Ernst Bottcher, Koln (DE); Iris Heep, Koln (DE); Georg Schulte, Wuppertal (DE); Ulrike Umgelder, Leverkusen (DE)

Correspondence Address: BAYER HEALTHCARE LLC, P.O.BOX 390, SHAWNEE MISSION, KS, 66201, US

(73) Assignee: BAYER HEALTHCARE AG, D-51368 LEVERKUSEN (DE)

(21) Appl. No.: 11/721,204

(22) PCT Filed: Dec. 3, 2005
(86) PCT No.: PCT/EP05/12978

§ 371 Date: Jul. 3, 2007

(30) Foreign Application Priority Data
Dec. 9, 2004 (DE) 10 2005 059 220.9
Nov. 17, 2005 (DE) 10 2005 055 385.0

(51) Int. Cl. A61K 33/38; A61K 31/5395; A61K 31/4164; A61K 31/56; A61K 31/57; A61K 31/58

(52) U.S. Cl. 424/618; 514/229.2; 514/396; 514/399; 514/171

(57) ABSTRACT

The invention relates to a system for hygienically administering, particularly in animals, an ear medicament which can be dosed reproducibly even in the case of low volumes and which is not flung out once again even when the head is shaken.

The invention relates to a system, as a pharmaceutical, for hygienically administering an ear medicament, in particular in animals, which can be metered in reproducibly even in the case of small volumes and which is not flung out once again even when the head is shaken.

Inflammation of the external auditory canals (otitis externa) can quite frequently be observed in dogs. Investigations carried out by Grono et al. (Grono L R: Otitis externa. In Kirk, R W (ed.): Current Veterinary Therapy VII. W.B. Saunders Company, Philadelphia, 1980) in the USA showed that the incidence corresponded to about 5-8% of all clinical admissions whereas, on the other hand, ear inflammations occur more rarely in the cat.

The reason for the complex course of the disease in the dog is usually a coincidence of predisposing factors (e.g. pendulous ears and a high level of cerumen production), primary factors (primary disease such as atopia or feedstuff allergy, or seborrhoea) and sustaining factors (multiplication of bacteria and yeast in the auditory canal), which factors lead to a vicious circle consisting of microbial growth, on the one hand, and inflammation on the other hand. This circle can be broken by means of local treatment with bactericidal agents, in connection with which it is advantageous also to employ yeast-destroying substances and, where appropriate, a corticoid which has an anti-inflammatory, anti-pruritic and detumescent effect and which reduces secretion.

Generally speaking, the administration of ear medicaments to animals is made more difficult by the fact that the animals frequently resist the treatment and, after treatment, attempt to remove the medicament, for example by means of shaking the head.

As a rule, after the veterinarian has diagnosed the condition and carried out the initial therapy, the otitis is treated by the owner. Problems which can delay, or call into question, the success of the treatment arise in this connection.
The dosage imprecision which arises as a result of lay persons using relatively large multiple dispensing containers for performing administration into the ear.
The problems of hygiene which arise as a result of secretion being aspirated into the container when the pressure is reduced prematurely or as a result of the top of the bottle making contact with secretion in the ear, and the contamination of the medicament which is caused in this way.
The unwieldy nature of the large multiple dispensing containers leads to uncertainty when lay persons are performing administrations into the tender ears.

The abovementioned factors also call into question consistent adherence to the therapy since, because of the problems, owners frequently carry out the treatment irregularly or imprecisely. When the administration of customary preparations from multiple dispensing containers takes place while being monitored visually, it can be seen that the drops also fall at the edge of the auditory canal, or even at the side of it, since the animal moves during the treatment. When the administration takes place without any visual monitoring, i.e. by means of inserting the bottle tops of customary multiple dispensing containers into the auditory canal, it is not possible to monitor the dose and the pressure which may possibly be applied during the administration can result in the already inflamed ear being injured.

The object of the invention was therefore to find a pharmaceutical which enables the ear to be treated with a precise dose in a hygienic and simple manner.

Extensive studies have been carried out on oily solutions or suspensions which are thickened with highly disperse silicon dioxide. These latter are offered for sale as a multidose container which is usually intended for oral use. While single dosage units have also already been described on a number of occasions, this is for oral use in the form of capsules (see, for example, U.S. Pat. No. 5,665,384, U.S. Pat. No. 4,450,877 or WO 00/33866). Thixotropic, oily formulations have also been disclosed (FR 2790200, WO 00/01371 and WO 03/022254).

While these documents describe oily, thixotropic formulations, either the latter are taken orally, in their entirety, in the form of capsules, thereby guaranteeing reproducible dosing, or the preparations are aliquoted into relatively large containers having higher contents of active compound (FR 2790200), with this likewise considerably facilitating reproducible dosing. The restoring force in the descriptions of the thixotropic formulations only serves the purpose of filling the capsules and not that of using the formulations in practice on a patient's ear (WO 00/01371).

The invention therefore relates to:

A pharmaceutical for treating diseases of the ear in humans or animals, comprising:
(a) an anti-infective agent
(b) in a liquid base
   aliquoted into a primary packaging means for once-only administration.

Anti-infective agents are, in particular, compounds such as penicillins, cephalosporins, aminoglycosides, sulphonamides and, in particular, quinolones, which exhibit antibacterial activity. Quinolones, preferably fluoroquinolones, are, inter alia, compounds as disclosed in the following documents: U.S. Pat. No. 4,670,444 (Bayer A G), U.S. Pat. No. 4,472,405 (Riker Labs), U.S. Pat. No. 4,730,000 (Abbott), U.S. Pat. No. 4,861,779 (Pfizer), U.S. Pat. No. 4,382,892 (Daiichi), U.S. Pat. No. 4,704,459 (Toyama); specific examples of quinolones which may be mentioned are pipemidic acid and nalidixic acid; examples of fluoroquinolones which may be mentioned are: benofloxacin, binfloxacin, cinoxacin, ciprofloxacin, danofloxacin, difloxacin, enoxacin, enrofloxacin, fleroxacin, ibafloxacin, levofloxacin, lomefloxacin, marbofloxacin, moxifloxacin, norfloxacin, ofloxacin, orbifloxacin, pefloxacin, temafloxacin, tosufloxacin, sarafloxacin and sparfloxacin.

A preferred group of fluoroquinolones are those of the formula (I) or (II):

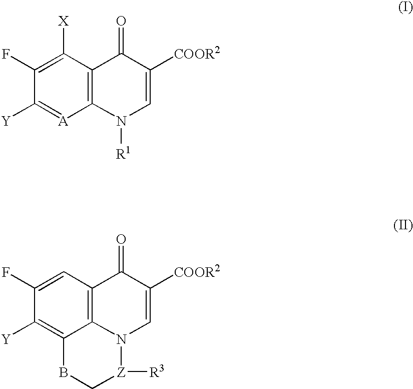

in which
X is hydrogen, halogen, C₁₋₄-alkyl, C₁₋₄-alkoxy, or NH₂,

Y is radicals of the structures

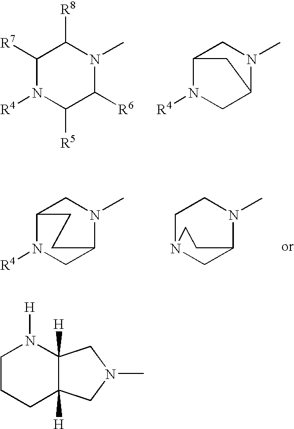

in which
R⁴ is optionally hydroxyl- or methoxy-substituted straight-chain or branched C₁-C₄-alkyl, cyclopropyl or acyl having 1 to 3 C atoms,
R⁵ is hydrogen, methyl, phenyl, thienyl or pyridyl,
R⁶ is hydrogen or C₁₋₄-alkyl,
R⁷ is hydrogen or C₁₋₄-alkyl,
R⁸ is hydrogen or C₁₋₄-alkyl,
   and also
R¹ is an alkyl radical having from 1 to 3 carbon atoms, cyclopropyl, 2-fluoroethyl, methoxy, 4-fluorophenyl, 2,4-difluorophenyl or methylamino,
R² is hydrogen or optionally methoxy- or 2-methoxyethoxy-substituted alkyl having from 1 to 6 carbon atoms and also cyclohexyl, benzyl, 2-oxopropyl, phenacyl, ethoxycarbonylmethyl or pivaloyloxymethyl,
R³ is hydrogen, methyl or ethyl, and
A is nitrogen, ═CH—, ═C(halogen)-, ═C(OCH₃)—, ═C(CH₃)— or ═C(CN),
B is oxygen, optionally methyl- or phenyl-substituted ═NH or ═CH₂,
Z is ═CH— or ═N—,
   and the pharmaceutically utilizable salts and hydrates thereof.

The compounds of the formulae (I) and (II) can be present in the form of their racemates or in enantiomeric forms.

Preference is given to compounds of the formula (I),

in which

### A is ═CH— or ═C—CN,

R¹ is optionally halogen-substituted C₁-C₃-alkyl or cyclopropyl,
R² is hydrogen or C₁₋₄-alkyl,
Y is radicals of the structures

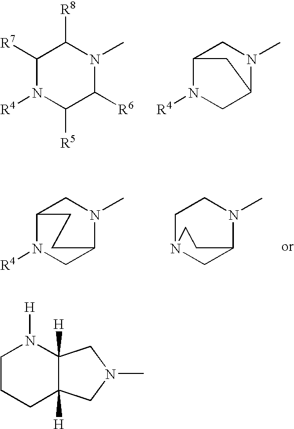

in which
R⁴ is optionally hydroxyl-substituted straight-chain or branched C₁-C₃-alkyl or oxalkyl having from 1 to 4 C atoms,
R⁵ is hydrogen, methyl or phenyl,
R⁷ is hydrogen or methyl,
R⁶ and R are hydrogen,
   and the pharmaceutically utilizable hydrates and salts thereof.

Particular preference is given to compounds of the formula (I), in which

### A is ═CH— or ═C—CN,

R¹ is cyclopropyl,
R² is hydrogen, methyl or ethyl,
Y is radicals of the structures

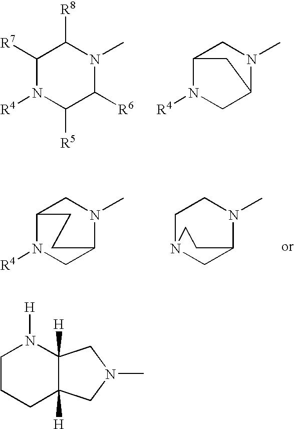

in which
R⁴ is methyl or optionally hydroxyl-substituted ethyl,
R⁵ is hydrogen or methyl,
R⁷ is hydrogen or methyl,
R⁶ and R⁸ are hydrogen,
   and the pharmaceutically utilizable salts and hydrates thereof.

Pharmaceutically utilizable acid addition salts and basic salts are suitable salts.

Pharmaceutically utilizable salts are to be understood, for example, as being the salts of hydrochloric acid, sulphuric acid, acetic acid, glycolic acid, lactic acid, succinic acid, citric acid, tartaric acid, methanesulphonic acid, 4-toluenesulphonic acid, galacturonic acid, gluconic acid, embonic acid, glutamic acid or aspartic acid. Furthermore, the compounds according to the invention can be bound to acidic or basic ion exchangers. Examples of pharmaceutically utilizable basic salts which may be mentioned are the alkali metal salts, for example the sodium or potassium salts, the alkaline earth metal salts, for example the magnesium or calcium salts, the zinc salts, the silver salts and the guanidinium salts.

Hydrates are understood as meaning both the hydrates of the fluoroquinolones themselves and the hydrates of the their salts.

Particularly preferred fluoroquinolones which may be mentioned are those described in WO 97/31001, in particular 8-cyano-1-cyclopropyl-7-((1S,6S)-2,8-diazabicyclo-[4.3.0]nonan-8-yl)-6-fluoro-1,4-dihydro-4-oxo-3-quinolinecarboxylic acid (pradofloxacin) of the formula

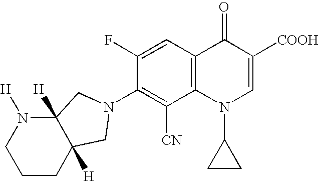

Pradofloxacin is preferably employed in its free form as the anhydrate, e.g. in the B modification (cf. WO 00/31076), or as the trihydrate (cf. WO 2005/097 789).

Particular preference is also given to using enrofloxacin:

1-Cyclopropyl-7-(4-ethyl-1-piperazinyl)-6-fluoro-1,4-dihydro-4-oxo-3-quinoline-carboxylic acid

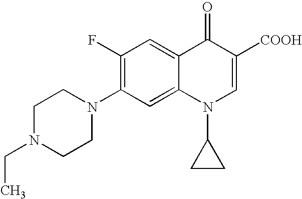

In addition to enrofloxacin and pradofloxacin, marbofloxacin, orbifloxacin, difloxacin and ibafloxacin may also be mentioned as being preferred quinolone anti-infective agents.

Examples of penicillins are benzylpenicillin, ampicillin, amoxicillin, oxacillin, piperacillin and ticarcillin.

Examples of cephalosporins are cefalexin, cefadroxil, cefazolin, cefoxitin and ceftiofur.

Examples of macrolides which may be mentioned are erythromycin, spiramycin, tylosin and tilmicosin.

Examples of sulphonamides which may be mentioned are trimethoprim and sulphadiazine (preferably used in combination).

Examples of aminoglycosides which may be mentioned are gentamicin, kanamycin, streptomycin, neomycin and spectinomycin.

Another antibiotic which may be mentioned is the lincosamide clindamycin. The anti-infective agent is typically employed in the formulation in a proportion of 0.001-6% by weight, preferably 0.01-1.0% by weight, particularly preferably 0.1-0.8% by weight.

Anti-infective agents which are less preferred within the meaning of this invention are derived from silver, e.g. colloidal silver, silver nitrate or silver sulphadiazine. However, these latter can be used in combination with one of the above-described anti-infective agents and/or, as described below, a corticoid where appropriate.

It is advantageous for the pharmaceutical according to the invention to comprise, in addition to the anti-infective agent and as a further pharmaceutically active constituent, an antimycotic agent such as an imidazole or a triazole, in particular, for example, clotrimazole, miconazole or bifonazole.

The antimycotic agent is typically employed in the formulation in a proportion of 0.01-10% by weight, preferably 0.1-5% by weight, particularly preferably 0.5-2% by weight.

It is furthermore advantageous for the pharmaceutical according to the invention to also comprise a corticoid in addition to the anti-infective agent and, where appropriate, the antimycotic agent. It is possible to employ both the corticoids and their derivatives, in particular the esters, which are customarily used for pharmaceutical purposes. Examples of corticoids which may be mentioned are hydrocortisone, prednisolone, betamethasone, mometasone and flumethasone; preferably betamethasone, triamcinolone and, in particular, dexamethasone.

In corticoid esters, the hydroxyl groups at C17 and/or C21 are usually esterified with short-chain organic acids; this increases the potency of the corticoids; the higher degree of lipophilia leads to better penetration into the cells and, at the same time, enrichment in the skin is improved. Thus, for example, hydrocortisone counts as one of the weak glucocorticoids while hydrocortisone-17-butyrate, on the other hand, counts as one of the strong glucocorticoids. Similar effects are to be observed in the case of the glucocorticoids dexamethasone/dexamethasone-21-acetate and betamethasone/betamethasone-17-valerate. Examples of corticoid esters are aclometasone propionate, betamethasone dipropionate, betamethasone valerate, clobetasol propionate, clobetasone butyrate, clocortolone hexanoate, clocortolone pivalate, dexamethasone aceatate, diflucortolone valerate, diflucortolone valerate, flumetasone pivalate, fluocortolone hexanoate, fluocortolone pivalate, fluprednidene acetate, fluticasone propionate, hydrocortisone butyrate, hydrocortisone aceponate, hydrocortisone acetate, hydrocortisone buteprate, methylprednisolone aceponate, mometasone furoate, prednicarbate and prednisolone acetate. Betamethasone-17-valerate and, in particular, dexamethasone-21-acetate are particularly preferred corticoid esters. Triamcinolone acetonide, a ketal, may be mentioned as being another particularly preferred example of a corticoid derivative.

Within the context of this invention, the term corticoid also encompasses, in its widest meaning, the derivatives such as the esters and ketals which were detailed above.

The corticoid is typically employed in the formulation in a proportion of 0.001-2.0% by weight, preferably 0.005-0.5% by weight, particularly preferably 0.05-0.2% by weight.

The following may be mentioned as being a particularly preferred active compound combination: pradofloxacin, clotrimazole and dexamethasone (preferably in the form of its 21-acetate).

As explained in more detail above with regard to the quinolones, it is possible, in the case of all the pharmaceutically active constituents, to use the corresponding pharmaceutically acceptable salts, hydrates and solvates and, where appropriate, different modifications.

Optically active substances can be used in the form of their stereoisomers or as stereoisomeric mixture, e.g. as pure or enriched enantiomers or as racemates.

The liquid base can be oily or aqueous.

Natural (animal or vegetable), synthetic and semisynthetic oils or fats can be used as the oily base. The oils or fats which are to be mentioned are soybean oil, sunflower oil, cottonseed oil, olive oil, groundnut oil, thistle oil, palm oil, rapeseed oil, coconut oil, maize-germ oil, castor oil and jojoba oil. Preference is given to using the medium-chain triglycerides (triglycerides containing saturated fatty acids, preferably octanoic acid and decanoic acid), propylene glycol diesters of caprylic acid/capric acid, low-viscosity paraffin or sesame oil; of these, particular preference is given to using the medium-chain triglycerides and propylene glycol diesters of caprylic acid/capric acid. These oils and fats can naturally also be employed as mixtures.

Water, glycerol, propylene glycol or polyethylene glycols can be used as the aqueous base. It is likewise possible to use mixtures of these substances.

An oily base is preferred.

The oily or aqueous base is typically employed in a proportion of 99.9-72% by weight, preferably of 99.4-89.5% by weight, particularly preferably of 97.9-94.0% by weight.

In the pharmaceuticals according to the invention, a liquid pharmaceutical formulation is aliquoted in a primary packaging means. In principle, the formulations can be solutions, emulsions, suspensions, pastes or gels.

The formulations can comprise thickeners, e.g. cellulose derivates such as methylcellulose, hydroxypropylcellulose, hydroxyethylcellulose, hydroxypropyl-methylcellulose, carboxymethylcellulose and microcrystalline cellulose; bentonites, kaolin, pectin, starches, modified starches, waxes, agar, paraffins, gelatin, alginates, polyvinylpyrrolidone, crospovidone, cetyl alcohol, stearates, such as magnesium sterarate, zinc stearate or glyceryl stearate, saturated or unsaturated long-chain (C₈-C₂₄) fatty acids, high molecular weight polyethylene glycols (e.g. polyethylene glycol 2000) or, preferably, silicon dioxides such as hydrophilic, precipitated, highly disperse, precompressed or hydrophobic, methylated silicon dioxides as well as mixed oxides composed of silicon oxide and aluminium oxide and, particularly preferably, highly disperse silicon dioxides.

The use of thickeners is, for example, advantageous when one or more active compounds does/do not dissolve, or does/do not dissolve adequately, in the liquid base such that a suspension has to be employed. The thickener then serves to stabilize the suspension against sedimentation.

The thickener is typically employed in the formulations in a proportion of 0.1-10% by weight, preferably of 0.5-5% by weight, particularly preferably of 1.0-3.0% by weight.

Preference is given to adjusting the formulation so as to ensure that it has thixotropic properties, meaning that its viscosity is reduced by shaking and builds up once again when the formulation is at rest. This results in it being readily possible to withdraw the formulation from the primary packaging means and for the formulation to be rapidly reconstituted such that the formulation which has been administered remains in the ear and cannot be flung out by means, for example, of the head being shaken. Thixotropic formulations are prepared by adding an appropriate additive to the formulation base (liquid base), provided the latter is not already itself thixotropic. An additive of this nature is usually a suspension stabilizer or thickener such as the highly disperse silicon dioxides or hydrophobic silicon dioxide (e.g. methylated silicon dioxide). The extent of the thixotropy can be adjusted deliberately by varying the concentration.

According to the invention, the primary packaging means are single dose containers. A volume of 0.1-5.0 ml, preferably 0.2-4.0 ml, particularly preferably 0.3-2.0 ml, of withdrawable content of liquid formulation is aliquoted into these containers.

The formulations can contain further customary, pharmaceutically tolerated additives and auxiliary substances. The following examples may be mentioned:
Preservatives, such as carboxylic acids (sorbic acid, propionic acid, benzoic acid or lactic acid), phenols (cresols, p-hydroxybenzoic esters such as methylparaben, propylparaben, etc.), aliphatic alcohols (benzyl alcohol, ethanol, butanol, etc.) or quaternary ammonium compounds (benzalkonium chloride or cetylpyridinium chloride).
Antioxidants such as sulphites (Na sulphite or Na metabisulphite), organic sulphides (cystine, cysteine, cysteamine, methionine, thioglycerol, thioglycolic acid or thiolactic acid), phenols (tocopherols, as well as vitamin E and vitamin E-TPGS (d-alpha-tocopherylpolyethylene glycol-1000-succinate)), butylhydroxyanisole, butylhydroxytoluene, gallic acid or its derivatives (propyl gallate, octyl gallate and dodecyl gallate), organic acids (ascorbic acid, citric acid, tartaric acid or lactic acid) and their salts and esters.
Wetting agents or emulsifiers such as fatty acid salts, fatty alkyl sulphates, fatty alkyl sulphonates, linear alkylbenzene sulphonates, fatty alkylpolyethylene glycol ether sulphates, fatty alkylpolyethylene glycol ethers, alkylphenolpolyethylene glycol ethers, alkylpolyglycosides, fatty acid-N-methylglucamides, polysorbates, sorbitan fatty acid esters, lecithins and poloxamers.
Pharmaceutically acceptable dyes such as iron oxides, carotenoids, etc.
The formulations can also comprise cosolvents which can furthermore also reduce the viscosity. These cosolvents are usually employed in proportions of from 0.1 to 40% by weight, preferably of from 1 to 10% by weight. The following cosolvents may be mentioned by way of example: pharmaceutically tolerated alcohols such as ethanol or benzyl alcohol, dimethyl sulphoxide, ethyl lactate, ethyl acetate, triacetin, N-methylpyrrolidone, glycerol formal, propylene carbonate, benzyl benzoate, glycofurol, dimethylacetamide, 2-pyrrolidone, isopropylidene glycerol, glycerol and polyethylene glycols. Mixtures of the abovementioned solvents can also be used as cosolvent.
Water.
Hexyldodecanol, decyl oleate, dibutyl adipate, dimethicone, glyceryl ricinoleate, octyldodecanol, octyl stearate, propylene glycol dipelargonate and, preferably, isopropyl myristate or isopropyl palmitate, can, inter alia, be employed as spreading agents.
Penetration enhancers (or permeation enhancers) improve the transdermal administration of pharmaceuticals and have in principle been disclosed in the prior art (see, for example, chapter 6 in Dermatopharmazie [Dermatopharmacy], Wissenschaftliche Verlagsgesellschaft [scientific publisher] mbH Stuttgart, 2001). Examples which may be mentioned are spreading oils such as isopropyl myristate, dipropylene glycol pelargonate, silicone oils or their copolymers with polyethers, fatty acid esters (e.g. oleyl oleate), triglycerides, fatty alcohols and linolene. DMSO, N-methylpyrrolidone, 2-pyrrolidone, dipropylene glycol monomethyl ether, octyldodecanol, oleyl macrogol glycerides or propylene glycol laurate can likewise be used.
It can furthermore be advantageous for the stability of the formulations if the latter comprise acids. In principle, inorganic and organic acids are suitable acids. Examples of inorganic acids are hydrochloric acid, sulphuric acid, sulphurous acid and phosphoric acid. Examples of organic acids are formic acid, acetic acid, propionic acid, butyric acid, lauric acid, palmitic acid, stearic acid, oleic acid, sorbic acid, citric acid, oxaloacetic acid, tartaric acid, methanesulphonic acid, lactic acid and ascorbic acid. Preference is given to using organic acids in oily bases in particular. Preferred examples are sorbic acid, stearic acid and propionic acid. Customary acid concentrations are in the range of up to 30% by weight, preferably of from 0.5 to 25% by weight, according to the nature of the formulation and the acid employed. In most cases, however, lower acid concentrations in the range of from 0.05 to 2% by weight, preferably of from 0.05 to 1% by weight, are usually employed.

The primary packaging means, a single dose container, usually has the form of a tube (hose tubes, laminate tubes, blow tubes or injection stretch tubes). The single dose containers can be made of polypropylene, polyethylene, aluminium (Al), of laminate, or of mixtures of these materials. The most frequently employed material for plastic tubes in general is at present polyethylene, specifically PE-LD (polyethylene-low density) and PE-HD (polyethylene-high density). Laminate tubes are multilayered tubes which are produced from aluminium oxide or silicon oxide (SiOx) and plastic coatings. The composites usually consist of PE-LD/AL/PE-LD and other layers. However, the aluminium layer can also be replaced with barrier-layer foils such as thermoplastics or barrier plastics, in particular with E/VAL (E/VOH; ethylene-vinyl alcohol) and silicon oxide (SiOx). Preference is given, according to the invention, to using tubes which are made of polyethylene, polypropylene or laminate, particularly preferably of laminate or, in particular, polypropylene.

Specially sterilizable tubes composed of polypropylene are, for example, tubes composed of PP/E/VAL/PP.

The tubes are opened by means of an unscrewable pin, a screw or push-pull closure with or without an additional sealing membrane, by means of a puncturable membrane including a spike, for example in the cap, by means of a peelable seal, for example in the form of a foil, or by means of a seal which can be broken off or torn off. Preference is given to the tubes being opened by means of a spike, which is located in the cap, for example, being screwed or stuck into the sealing membrane of the tube. The application tip should have a certain length even in the opened state and should be rounded at the front end in order to avoid injury.

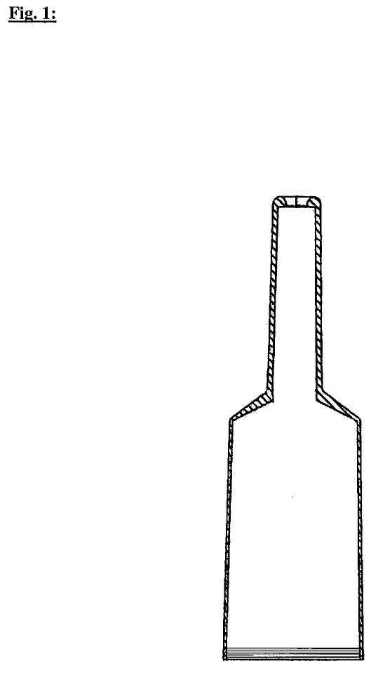

By way of example, FIG. 1 shows a tube which is suitable for use as a single dose container according to the invention.

When aliquoted into single dose containers, the described formulations are particularly well suited for treating otitis externa in dogs and cats in a hygienic manner. It is particularly to be emphasized that the formulation can be dispensed in a readily reproducible manner. The use of thickeners in suspension formulations can as a rule prevent any sedimentation of the suspended constituents. Thixotropic formulations are particularly advantageous since, after the single dose containers have been shaken, the formulation can be dispensed in a manner which is particularly readily reproducible, even at low active compound concentrations, and, as a result of the thixotropy and using the single-dose container, the formulation can be administered simply and hygienically into the ear of the animal and can nevertheless not be flung out by, for example, the customary shaking of the head. It is likewise desirable for the formulation to have good spreading behaviour since the formulation should become well dispersed in the auditory canal after it has been administered.

The formulations are produced by the active compounds or auxiliary substances which are to be dissolved or suspended being dispersed in the base. Where appropriate a mixing apparatus, or preferably a homogenizer or high-pressure homogenizer, is employed for the dispersing. The sequence in which the individual constituents are added can be varied in accordance with the formulation. After all the formulation constituents have been dispersed, the finished formulation is stored temporarily or aliquoted directly into the single-dose containers, which are then sealed.

The pharmaceuticals according to the invention are suitable in a general manner for being used in humans and animals. They are preferably employed in animal husbandry and animal breeding for productive animals, breeding animals, zoo animals, laboratory animals, experimental animals and domestic animals, and specifically for mammals, in particular.

The productive and breeding animals include mammals such as cattle, horses, sheep, pigs, goats, camels, water buffalo, donkeys, rabbits, fallow deer, reindeer and furred animals such as mink, chinchilla and racoon, as well as birds such as domestic fowl, geese, turkeys, ducks, pigeons and ostriches. Examples of preferred productive animals are cattle, sheep, pigs and domestic fowl.

The laboratory and experimental animals include dogs, cats, rabbits and rodents such as mice, rats, guinea pigs and golden hamsters.

The domestic animals include dogs, cats, horses, rabbits, rodents such as golden hamsters, guinea pigs and mice and, in addition, reptiles, amphibia and birds for being kept domestically and in zoos.

Preference is given to using the pharmaceuticals according to the invention for domestic animals and, in particular, for dogs and cats.

The pharmaceuticals can be used both prophylactically and therapeutically.

The formulations which are described here are envisaged for local administration into the auditory canals. However, other areas of application, such as dermal, oral, rectal, vaginal or nasal administration, are possible in principle.

### EXAMPLES

The percentage values for the formulations which are described here are given in weight per volume (grams of the relevant substance per 100 ml of finished formulation). The triglycerides of the caprylic/capric acid esters, for example Miglyol® 812 from Sasol/Witten (e.g. used in Examples 3 and 6), are to be used as medium-chain triglycerides.

### Example 1

0.15% pradofloxacin

0.05% betamethasone-17-valerate

0.5% bifonazole

2.0% highly disperse silicon dioxide

propylene glycol octanoate decanoate to make up to 100%

0.5 g of betamethasone valerate is suspended, together with 1.5 g of pradofloxacin and 5 g of bifonazole, in 973 g of propylene glycol octanoate decanoate, after which 20 g of highly disperse silicon dioxide are added. The suspension is then homogenized with a homogenizer for 10 min.

### Example 2

0.5% enrofloxacin

0.1% triamcinolone acetonide

1.0% clotrimazole

1.6% highly disperse silicon dioxide

medium-chain triglycerides to make up to 100%

10 g of enrofloxacin are suspended, together with 2 g of triamcinolone acetonide and 20 g of clotrimazole, in 1932 g of medium-chain triglycerides after which 36 g of highly disperse silicon dioxide are added. The suspension is then homogenized with a homogenizer for 10 min.

### Example 3

0.3% pradofloxacin (trihydrate)

0.1% dexamethasone-21-acetate

1.0% clotrimazole

1.8% highly disperse silicon dioxide

medium-chain triglycerides to make up to 100%

5 g of clotrimazole and 0.5 g of dexamethasone acetate are suspended, together with 1.5 g of pradofloxacin (calculated without water of hydration), in 484 g of MCT after which 9 g of highly disperse silicon dioxide are added. The suspension is then homogenized with a homogenizer for 10 min.

### Example 4

0.3% pradofloxacin

0.1% dexamethasone 21-acetate

1.0% clotrimazole

0.8% hydroxyethylcellulose

20% lactic acid

19% isopropanol

1.6% benzyl alcohol

propylene glycol to make up to 100%

200 g of isopropanol and 16 g of benzyl alcohol are mixed in 500 g of propylene glycol. 1 g of dexamethasone acetate, 3 g of pradofloxacin and 10 g of clotrimazole are suspended in this mixture, after which 200 g of lactic acid are added. 8 g of hydroxyethylcellulose are stirred and 62 g of propylene glycol are used to make up to the final weight. The suspension is then homogenized with a homogenizer for 10 min.

### Example 5

0.15% marbofloxacin

0.05% triamcinolone acetonide

0.5% bifonazole

0.05% propyl gallate

1.7% highly disperse silicon dioxide

propylene glycol octanoate decanoate to make up to 100%

0.15 g of propyl gallate is suspended in 1427.85 g of propylene glycol octanoate decanoate. 1.5 g of triamcinolone acetonide, 15 g of bifonazole and 4.5 g of marbofloxacin are suspended in this dispersion after which 51 g of highly disperse silicon dioxide are added. The suspension is then homogenized with a homogenizer for 10 min.

### Example 6

0.3% pradofloxacin (trihydrate)

0.03% dexamethasone 21-acetate

1.0% clotrimazole

1.8% highly disperse silicon dioxide

medium-chain triglycerides to make up to 100%

3 g of pradofloxacin (calculated without water of hydration) are suspended, together with 0.3 g of dexamethasone acetate and 10 g of clotrimazole, in 968.7 g of medium-chain triglycerides after which 18 g of highly disperse silicon dioxide are added. The suspension is then homogenized with a homogenizer for 10 min.

### Example 7

0.3% pradofloxacin

0.03% dexamethasone 21-acetate

1.0% clotrimazole

0.1% propyl gallate

2.3% highly disperse silicon dioxide

1.0% vitamin E

sesame oil to make up to 100%

1 g of propyl gallate is dispersed in 952.7 g of sesame oil after which 0.3 g of dexamethasone acetate, 10 g of clotrimazole and 3 g of pradofloxacin are suspended in this dispersion. The mixture is then supplemented with 10 g of vitamin E 18 g and 23 g of highly disperse silicon dioxide. The suspension is then homogenized with a homogenizer for 10 min.

### Example 8

0.5% enrofloxacin

0.1% dexamethasone 21-acetate

1.0% bifonazole

2% n-butanol

1.9% highly disperse silicon dioxide

medium-chain triglycerides to make up to 100%

0.5 g of n-butanol is mixed in 241 g of medium-chain triglycerides. 0.25 g of dexamethasone acetate, 1.25 g of enrofloxacin and 2.5 g of bifonazole are dispersed in this mixture, to which 4.5 g of highly disperse silicon dioxide are then added. The suspension is then homogenized with a homogenizer for 10 min.

### Example 9

0.3% pradofloxacin

0.1% betamethasone 17-valerate

1.0% clotrimazole

0.01% BHT

2.0% highly disperse, hydrophobic silicon dioxide

jojoba oil to make up to 100%

1 g of BHT is suspended in 9.7 kg of jojoba oil after which 10 g of betamethasone valerate, 30 g of pradofloxacin, 180 g of highly disperse silicon dioxide and 100 g of clotrimazole are suspended therein. The suspension is then homogenized with a homogenizer for 10 min.

### Example 10

0.114% pradofloxacin trihydrate

0.05% dexamethasone 21-acetate

0.5% clotrimazole

0.1% sorbic acid

1.8% highly disperse silicon dioxide

medium-chain triglycerides to make up to 100%

0.1 kg of sorbic acid, 0.5 kg of clotrimazole and 0.05 kg of dexamethasone 21-acetate are dissolved in 92.8 kg of medium-chain triglycerides. 0.114 kg of pradofloxacin trihydrate and 1.8 kg of highly disperse silicon dioxide are dispersed in this solution. The suspension is then homogenized with a homogenizer for 10 min.

### Example 11

0.114% pradofloxacin trihydrate

0.05% dexamethasone 21-acetate

0.5% clotrimazole

0.1% sorbic acid

1.7% highly disperse silicon dioxide

medium-chain triglycerides to make up to 100%

0.1 kg of sorbic acid, 0.5 kg of clotrimazole and 0.05 kg of dexamethasone 21-acetate are dissolved in 70 kg of medium-chain triglycerides. 0.114 kg of pradofloxacin trihydrate and 1.7 kg of highly disperse silicon dioxide are dispersed in this solution, which is supplemented with the remaining medium-chain triglycerides (22.9 kg). The suspension is then homogenized with a homogenizer for approx. 10 min.

### Example 12

0.114% pradofloxacin trihydrate

0.05% dexamethasone 21-acetate

0.5% clotrimazole

0.1% sorbic acid

3.6% methylated silicon dioxide (Aerosil® R 972, dimethyldichlorosilane-hydrophobized pyrogenic silicic acid from Degussa)

medium-chain triglycerides to make up to 100%

0.1 kg of sorbic acid, 0.5 kg of clotrimazole and 0.05 kg of dexamethasone 21-acetate are dissolved in 95.64 kg of medium-chain triglycerides. 0.114 kg of pradofloxacin trihydrate and 3.6 kg of hydrophobic silicon dioxide are dispersed in this solution. The suspension is then homogenized with a homogenizer for approx. 10 min.

### Example 13

0.114% pradofloxacin trihydrate

0.05% dexamethasone 21-acetate

0.5% clotrimazole

0.1% sorbic acid

2.7% methylated silicon dioxide (Aerosil® R 974, dimethyldichlorosilane-hydrophobized pyrogenic silicic acid from Degussa)

medium-chain triglycerides to make up to 100%

0.1 kg of sorbic acid, 0.5 kg of clotrimazole and 0.05 kg of dexamethasone 21-acetate are dissolved in 96.66 kg of medium-chain triglycerides. 0.114 kg of pradofloxacin trihydrate and 2.7 kg of hydrophobic silicon dioxide are dispersed in this solution. The suspension is then homogenized with a homogenizer for approx. 10 min.

1. A pharmaceutical for treating diseases of the ear in humans or animals, comprising:
(a) an anti-infective agent
(b) in a liquid base
aliquoted in a primary packaging means for once-only administration.

2. Pharmaceutical according to claim 1, comprising a fluoroquinolone.

3. Pharmaceutical according to claim 2, comprising enrofloxacin.

4. Pharmaceutical according to claim 2, comprising pradofloxacin.

5. Pharmaceutical according to claim 2, comprising marbofloxacin.

6. Pharmaceutical according to claim 1, additionally comprising another substance having an anti-infective effect, such as colloidal silver, silver nitrate or silver sulphadiazine.

7. Pharmaceutical according to claim 1, additionally comprising an antimycotic agent.

8. Pharmaceutical according to claim 7, comprising clotrimazole, miconazole or bifonazole.

9. Pharmaceutical according to claim 1, additionally comprising a corticoid.

10. Pharmaceutical according to claim 9, comprising dexamethasone, betamethasone or triamcinolone (or their derivatives).

11. Pharmaceutical according to claim 10, comprising dexamethasone 21-acetate.

12. Pharmaceutical according to claim 1, in which the content of the primary packaging means exhibits thixotropic properties.

13. Pharmaceutical according to claim 1, in which the content of the primary packaging means exhibits an oily liquid base.

14. Pharmaceutical according to claim 1, in which the content of the primary packaging means is a suspension.

15. Pharmaceutical formulation comprising
(i) from 0.001 to 6% by weight of an anti-infective agent
(ii) from 0.01 to 10% by weight of an antimycotic agent
(iii) from 0.001 to 2% by weight of a corticoid
(iv) from 99.9 to 72% by weight of a liquid base.

16. Pharmaceutical formulation according to claim 15, comprising, as corticoid, a corticoid ester.

17. Pharmaceutical formulation according to claim 15, characterized by an oily liquid base.

18. Pharmaceutical formulation according to one of claims 15, comprising an acid.

19. Pharmaceutical formulation according to claim 18, comprising an organic acid, in particular sorbic acid, stearic acid and propionic acid.

20. Pharmaceutical formulation according to claim 18, comprising up to 30% by weight of acid.

21. Pharmaceutical formulation according to claim 18, comprising from 0.05 to 2% by weight of acid.
